(19) United States
(12) Patent Application Publication
(10) Pub. No.: US 2004/0034041 A1
(43) Pub. Date: Feb. 19, 2004

(54) Novel anti-infectives

(76) Inventors: Dashyant Dhanak, Collegeville, PA (US); Arun C. Kaura, Collegeville, PA (US); Atony Shaw, Collegeville, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/275,581

(22) PCT Filed: May 10, 2001
(86) PCT No.: PCT/US01/15105

(51) Int. Cl. A61K 31/52

(52) U.S. Cl. 514/263.1

(57) ABSTRACT

Novel anti-infectives and methods of using them are provided.

### FIELD OF THE INVENTION

The present invention relates to novel anti-infectives. Specifically, the present invention involves novel HCV inhibitors.

### BACKGROUND OF THE INVENTION

First identified by molecular cloning in 1989 (Choo et al., 1989), hepatitis C virus (HCV) is now widely accepted as the most common causative agent of post-transfusion non A, non-B hepatitis (NANBH) (Kuo et al., 1989). Due to its genome structure and sequence homology, this virus was assigned as a new genus in the Flaviviridae family, along with the other two genera, flaviviruses (such as yellow fever virus and Dengue virus types 1-4) and pestiviruses (such as bovine viral diarrhea virus, border disease virus, and classic swine fever virus) (Choo et al., 1989; Miller and Purcell, 1990). Like the other members of the Flaviviridae, HCV is an enveloped virus containing a single strand RNA molecule of positive polarity. The HCV genome (see FIG. 1) is approximately 9.6 kilobases (kb) with a long, highly conserved, noncapped 5′ nontranslated region (NTR) of approximately 340 bases which functions as an internal ribosome entry site (IRES) (Wang and Siddiqui, 1995). This element is followed by a region which encodes a single long open reading frame (ORF) encoding a polypeptide of ˜3000 amino acids comprising both the structural and nonstructural viral proteins. Upon entry into the cytoplasm of the cell, this RNA is directly translated into a polypeptide of ˜3000 amino acids comprising both the structural and nonstructural viral proteins (see FIG. 2.1). This large polypeptide is subsequently processed into the individual structural and nonstructural proteins by a combination of host and virally-encoded proteinases (reviewed in Rice, 1996). Following the termination codon at the end of the long ORF, there is a 3′ NTR which roughly consists of three regions: an 40 base region which is poorly conserved among various genotypes, a variable length poly(U)/polypyrimidine tract, and a highly conserved 98 base element also called the “3′X-tail” (Kolykhalov et al., 1996; Tanaka et al., 1995; Tanaka et al., 1996; Yamada et al., 1996). The 3′ NTR is predicted to form a stable secondary structure which is essential for HCV growth in chimps and is believed to function in the initiation and regulation of viral RNA replication.

Infection with HCV is a major cause of human liver disease throughout the world with seroprevalence in the general population ranging from 0.3 to 2.2% (van der Poel et al., 1994) to as high as 10-20% in Egypt (Hibbs et al., 1993). HCV is most commonly transmitted via blood (Alter et al., 1993). Of these initial infections, an estimated 30% are symptomatic. However, more than 85% of all infected individuals become chronically infected (3.9 million current chronic infections in US, 170 million chronic infections worldwide, estimated 33,200 new cases in 1994 in US). Chronic HCV infection accounts for 30% of all cirrhosis, end-stage liver disease, and liver cancer in the U.S. Of the total chronic cases in the US, greater than 118,000 will go on to develop hepatocellular carcinoma (HCC) (which represents≧25% of all liver cancers) as a direct result of HCV infection (reviewed in Hoofnagle, 1997; Seeff, 1997). There are 8,000-12,000 deaths per year in the US currently attributed to HCV infection, and treatment costs were estimated at 600 million for 1992 in the US. The CDC estimates that the number of deaths due to HCV will increase to 38,000/yr. by the year 2010.

Due to the high degree of variability in the viral surface antigens, existence of multiple viral genotypes, and demonstrated specificity of immunity, the development of a successful vaccine in the near future is unlikely. Although initially therapy consisted of interferon alone, combination therapy of interferon alpha-2b (●-IFN, 3 million units injected subcutaneously three times weekly) with ribavirin (1-1.2-gms twice daily orally) for either 24 or 48 weeks is currently the most efficacious approved therapy for the treatment of chronic HCV infection. Schering-Plough alone reported over $430 million in sales for interferon alone in 1998 specifically for HCV therapy. The response and sustained response rates for combination therapy were better than interferon alone (80% initial response for combo vs. 46% for IFN alone; and 30-50% sustained response for combo vs. 5-13% for IFN alone). However, there were still many adverse side effects associated with combination therapy (flu-like symptoms, leukopenia, thrombocytopenia, depression, etc. from interferon), as well as anemia induced by ribavirin (reviewed in Lindsay, 1997). Furthermore, this therapy was still less effective against infections caused by HCV genotype 1 which constitutes ˜75% of all HCV infections in the developed markets (as opposed to other HCV genotypes). Analogous to therapy for HIV infection, combination therapy (i.e. IFN plus antiviral or antiviral cocktail) is likely, to be the most efficacious therapy.

In the US, an estimated 3.9 million Americans are infected with HCV. Although only 30% of acute infections are symptomatic, >85% of infected individuals develop chronic, persistent infection. There are 8,000-10,000 deaths per year in the US currently attributed to HCV infection, and treatment costs are estimated at >600 million/yr. (1992 CDC estimate for US). Worldwide over 170 million people are estimated to be infected chronically. HCV infection is responsible for 40-60% of all chronic liver disease and 30% of all liver transplants. A vaccine is unlikely due to hypervariable surface antigens and demonstrated specificity of immunity.

Currently, there are no HCV antiviral agents available, with alpha-interferon (alone or in combination with ribavirin) being the only approved treatment. Many adverse side effects are associated with therapy (flu-like symptoms, leukopenia, thrombocytopenia, depression, anemia, etc.); only 50-80% of the patients respond (reduction in serum HCV RNA levels, normalization of liver enzymes); however, of those treated, 50-70% relapse within 6 months of cessation of therapy.

The NS5B protein (591 amino acids, 65 kDa) of HCV (Behrens et al., 1996), encodes an RNA-dependent RNA polymerase (RdRp) activity and contains canonical motifs present in other RNA viral polymerases. The NS5B protein is fairly well conserved both intratypically (one type 1b isolate vs. another type 1b isolate, 95-98% aa identity) and intertypically (type 1a vs. type 1b, 85% aa identity). The essentiality of the HCV NS5B RdRp activity for the generation of infectious progeny virions has been formally proven in chimpanzees (A. A. Kolykhalov et al. abstract, 1999) and inhibition of NS5B RdRp activity is therefore predicted to be antiviral for HCV infection, and inhibition of RNA replication would be expected to cure infection.

Based on the foregoing, there exists a significant need to identify synthetic or biological compounds for their ability to inhibit HCV.

### SUMMARY OF THE INVENTION

The present invention involves compounds represented hereinbelow, pharmaceutical compositions comprising such compounds and methods of using the present compounds.

### DETAILED DESCRIPTION OF THE INVENTION

The compounds useful in the present methods are selected from Formula (I) hereinbelow:

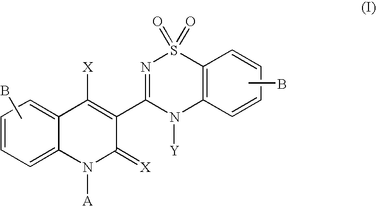

wherein:

A is selected from the group consisting of C 1-6 alkyl, C 2-6 alkenyl, alkylaryl, aryl, and heteroaryl;

B is selected from one or more of the group consisting of H, C 1-6 alkyl, C 1-6 cycloalkyl, halo, OR1, COR1, COOR1, CONR1R2, and CN wherein R1 and R2 are, independently, H, C 1-6 alkyl, aryl and heteroaryl;

X is selected from the group consisting of O, OR1, S, and SR1 wherein R1 is as defined above; and

Y is selected from the group consisting of H, C 1-6 alkyl, alkylaryl, aryl, and heteroaryl.

Preferably, A is selected from the group consisting of C 1-6 alkyl, and alkylaryl;

Preferably, B is H.

Preferably, X is selected from the group consisting of OH, and SH.

Preferably, Y is hydrogen.

As used herein, “alkyl” refers to an optionally substituted hydrocarbon group joined together by single carbon-carbon bonds. The alkyl hydrocarbon group may be linear, branched or cyclic, saturated or unsaturated.

As used herein, “aryl” refers to an optionally substituted aromatic group with at least one ring having a conjugated pi-electron system, containing up to two conjugated or fused ring systems. “Aryl” includes carbocyclic aryl, heterocyclic aryl and biaryl groups, all of which may be optionally substituted. Preferred aryl moieties are phenyl, unsubstituted, monosubstituted, disubstituted or trisubstituted. Preferred heteroaryl moieties are selected from the group consisting of unsubstituted, monosubstituted, disubstituted or trisubstituted thienyl, quinolinyl, indolyl and pyridinyl. Preferred aryl and heteroaryl substituents are selected from the group consisting of C 1-4 alkyl, NC 1-4 alkyl, halo, OC1-4 alkyl, CH═CH, CF3, pyridine, phenyl, NO2, CN, OH and MeO.

More preferably, alkyl substituents are methyl or ethyl. More preferably, halo substituents are chloro or bromo.

Preferred compounds useful in the present invention are selected from the group consisting of:

1-(n-Propyl)-3-(1,1-dioxo-2H-benzo-1,2,4-thiadiazin-3-yl)-4-hydroxy-2-quinolone;

1-(n-Butyl)-3-(1,1-dioxo-2H-benzo-1,2,4-thiadiazin-3-yl)-4hydroxy-2-quinolone;

1-Benzyl-3-(1,1-dioxo-2H-benzo-1,2,4-thiadiazin-3-yl)-4-hydroxy-2-quinolone;

1-(2-Pyridylmethyl)-3-(1,1-dioxo-2H-benzo-1,2,4-thiadiazin-3-yl)-4-hydroxy-2-quinolone;

1-(3-Cyanopropyl)-3-(1,1-dioxo-2H-benzo-1,2,4-thiadiazin-3-yl)-4-hydroxy-2-quinolone;

1-[(3-Methyl)butyl)-3-(1,1-dioxo-2H-benzo-1,2,4-thiadiazin-3-yl)-4-hydroxy-2-quinolone

1-[(2-Methyl)propyl)-3-(1,1-dioxo-2H-benzo-1,2,4-thiadiazin-3-yl)-4-hydroxy-2-quinolone

1-(4-Cyanobutyl)-3-(1,1-dioxo-2H-benzo-1,2,4-thiadiazin-3-yl)-4-hydroxy-2-quinolone;

1-(n-Pentyl)-3-(1,1-dioxo-2H-benzo-1,2,4-thiadiazin-3-yl)-4-hydroxy-2quinolone; and

1-(2-Butenyl)-3-(1,1-dioxo-2H-benzo-1,2,4-thiadiazin-3-yl)-4-hydroxy-2-quinolone.

1-(2-Propenyl)-3-(1,1-dioxo-2H-benzo-1,2,4-thiadiazin-3-yl)-4hydroxy-2-quinolone.

Also included in the present invention are pharmaceutically acceptable salt complexes. Preferred are the ethylene diamine, sodium, potassium, calcium, ethanolamine, hydrochloride, hydrobromide and trifluoroacetate salts.

Also included in the present invention is a process according to Scheme 1 for the synthesis of the compounds:

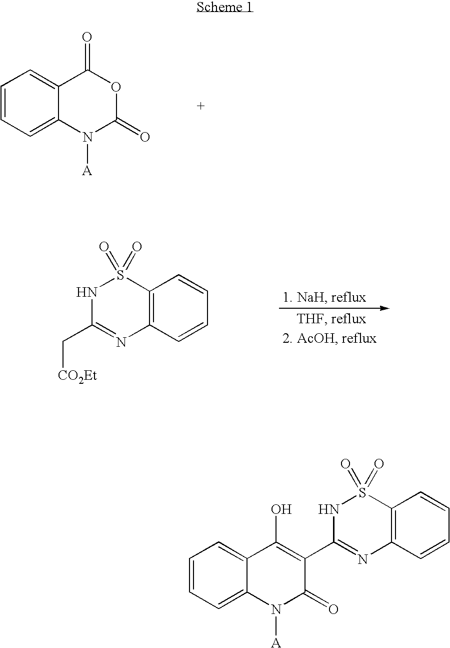

Also included in the present invention is a process according to Scheme 2 for the synthesis of the compounds:

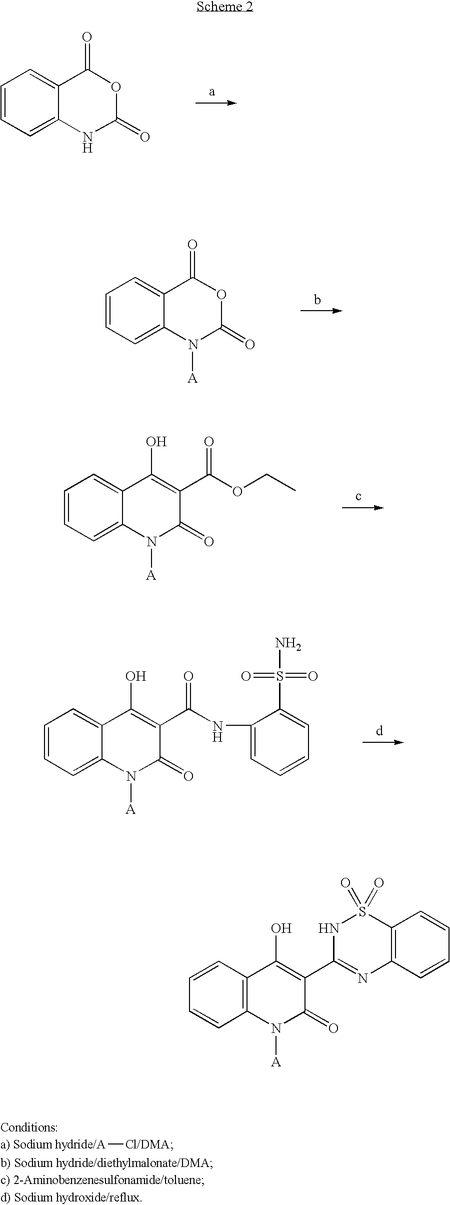

With appropriate manipulation and protection of any chemical functionality, synthesis of the remaining compounds of Formula (I) is accomplished by methods analogous to those above and to those described in the Experimental section.

### EXAMPLE 1

1-(3-Cyanopropyl)-3-(1,1-dioxo-2H-benzo-1,2,4-thiadiazin-3-yl-4-hydroxy-2-quinolone

a) N-(3-cyanopropyl)isatoic anhydride

Isatoic anhydride (1.63 g, 10.0 mmol) was added to a stirred mixture of 4-bromobutyronitrile (2.96 g, 20.0 mmol), potassium carbonate (4.14 g, 30.0 mmol) and dimethylformamide (15 ml) and stirring continued for 5 h. The mixture was poured into water and extracted with ethyl acetate. The extracts were washed with water, saturated aqueous NaCl, and dried (MgSO4). The solvent was removed under reduced pressure and the residue chromatographed (silica gel, 50-70% ethyl acetate/hexane) to give the title compound (187 mg, 8%) as a gum.

b) 1-(3-Cyanopropyl)-3-(1,1-dioxo-2H-benzo-1,2,4-thiadiazin-3-yl)-4-hydroxy-2-quinolone

Sodium hydride (60 mg of a 60% oil dispersion, 1.50 mmol) was added to a stirred solution of N-(3-cyanopropyl)isatoic anhydride (86 mg, 0.373 mmol) and ethyl 1,1-dioxo-2H-benzo-1,2,4-thiadiazinyl-3-acetate (100 mg, 0.373 mmol, lit. ref.: Kovalenko, S. N.; Chemykh, V. P.; Shkarlat, A. E.; Ukrainets, I. V.; Gridasov, V. I.; Rudnev, S. A., Chem.Heterocycl.Compd.(Engl.Transl.), 1998, 34(7), 791) in tetrahydrofuran (3 mL) at room temperature under argon. After 5 min, the mixture was stirred under reflux for 2.5 h, then cooled and acetic acid (1 mL) added. The mixture was heated again under reflux for 1 h, then cooled, poured into aqueous hydrochloric acid and cooled in ice. The precipitate was filtered, washed with water and air-dried. The solid was reprecipitated from tetrahydrofuran/water to give the title compound (100 mg, 66%) as a pale yellow solid. LCMS m/z 409 [M+H]+.

### EXAMPLE 2

3-(1,1-Dioxo-1,4-dihydro-1l6-benzo[1,2,4]thiadiazin-3-yl)-4-hydroxy-1-pyridin-2-ylmethyl-1H-Quinolin-2-one

a) 1-Pyridin-2-ylmethyl-1H-benzo[d][1,3]oxazin-2,4-dione

To a suspension of isatoic anhydride (1.63 g, 10 mmol) in anhydrous dimethylacetamide (10 ml) was added sodium hydride (0.44 g, 11 mmol, 60% oil dispersion) in portions. After stirring at room temperature for 30 minutes, 2-chloromethylpyridine [prepared from treatment of a suspension of 2-picolyl chloride hydrochloride (2.16 g, 13.17 mmol) with aqueous base, drying (MgSO4) and subsequent evaporation of organic solvent] was added and stirring continued for 48 hrs. The mixture was poured into iced water (100 ml), stirred vigorously for 30 minutes and the resulting brown solid filtered off. After air-drying, the yield was 1.05 g, 41%. MS (ES+) m/e 255 [M+H]+, and 509 [2M+H]+.

b) 4-Hydroxy-2-oxo-1-pyridin-2-ylmethyl-1,2-dihydroquinolin-3-carboxylic acid ethyl ester

Sodium hydride (0.33 g, 8.27 mmol, 60% oil dispersion) was washed with n-hexane (2×25 ml), dried off in vacuo, and re-suspended in anhydrous dimethylacetamide (5 ml). This suspension was stirred and treated portion-wise with diethyl malonate (1.32 g, 8.27 mmol) in anhydrous dimethylacetamide (5 ml) at room temperature. After 15 minutes, a solution of 1-pyridin-2-ylmethyl-1H-benzo[d][1,3]oxazin-2,4-dione (1.05 g, 4.13 mmol) was added over 2 minutes and the mixture warmed to 120° C. After another 2 hrs. the mixture was cooled, concentrated to a quarter of the original volume. The residue was partitioned between dichloromethane (25 ml) and water (20 ml), the aqueous phase separated, washed with more dichloromethane (4×25 ml), and acidified. The aqueous phase was extracted with fresh dichloromethane (3×25 ml), combined organic extracts dried (MgSO4) and evaporated to afford a solid (0.86 g) which was washed successively with ethanol and diethyl ether to give the title compound as pale brown crystals (0.37 g, 28%). 1H NMR (CDCl3)−14.4 (1H, s), 8.6 (1H, m), 8.2 (1H, dd), 7.5-7.6 (2H, m), 7.4 (1H, d), 7.1-7.3 (together 3H, m), 5.6 br (2H, s), 4.55 (2H, q), and 1.65 (2H, t), MS (ES+) m/e 325 [M+H]+, and 671 [2M+H]+.

c) 4-Hydroxy-2-oxo-1-pyridin-2-ylmethyl-1,2-dihydroquinolin-3-carboxylic acid (2-sulfamoylphenyl)-amide

4-Hydroxy-2-oxo-1-pyridin-2-ylmethyl-1,2-dihydroquinolin-3-carboxylic acid ethyl ester (213 mg, 0.657 mmol) was dissolved in toluene (10 ml) at 50° C. and 2-aminobenzenesulfonamide (113 mg, 0.656 mmol) added. The resulting mixture was heated under reflux for 18 hrs., cooled to room temperature and the solid formed filtered off. After washing well with diethyl ether the solid was dried in vacuo (290 mg, 98%). MS (ES+) m/e 451 [M+H]+, and 901 [2M+1]+.

d) 3-(1,1-Dioxo-1,4-dihydro-1l6-benzo[1,2,4]thiadiazin-3-yl)-4-hydroxy-1-pyridin-2-ylmethyl-1H-quinolin-2-one

4-Hydroxy-2-oxo-1-pyridin-2-ylmethyl-1,2-dihydroquinolin-3-carboxylic acid (2-sulfamoylphenyl)-amide (115 mg, 2.3 mmol) was dissolved in 10% aqueous NaOH (4 ml), water (10 ml) added and the resulting solution heated under reflux for 2 days. After cooling, the solution was filtered, filtrate acidified and the milky solution filtered. The solid thus obtained was heated in DMSO (2 ml) and the hot suspension filtered. The filtrate was purified by preparative HPLC to give the title compound (20 mg, 18%). 1H NMR (DMSO) 15.4 br(1H,s), 14.25br (1H, s), 8.5 (1H, d), 8.25 (1H, d), 7.95 (1H, d), 7.75-7.85 (together 3H, m), 7.7 (1H, d), 7.5-7.6 (together 2H, m), 7.45 (1H, t), 7.35 (1H, d), 7.3 (1H, m), and 5.75 (2H, s). MS (ES+) m/e 433 [M+H]+.

### EXAMPLE 3

1-Benzyl-3-(1,1-dioxo-1,2-dihydro-1l6-benzo[1,2,4]thiadiazin-3-yl)-4-hydroxy-1H-quinolin-2-one

Following the procedure of Example 2(a), 1(b), 1(c) and 1(d), except substituting benzyl bromide for 2-chloromethylpyridine, the title compound was prepared as a white crystalline solid. MS (ES+) m/e 432 [M+H]+.

### EXAMPLE 4

1-Butyl-3-(1,1-dioxo-1,2-dihydro-1l6-benzo[1,2,4]thiadiazin-3-yl)-4-hydroxy-1H-quinolin-2-one

Following the procedure of Example 1(a), 1(b), 1(c) and 1(d), except substituting n-butyl bromide for 2-chloromethylpyridine, the title compound was prepared as a white crystalline solid. MS (ES+) m/e 398 [M+H]+, 795 [2M+1]+, and 1214 [3M+Na]+.

### EXAMPLE 5

1-[(3-Methyl)butyl)-3-(1,1-dioxo-2H-benzo-1,2,4-thiadiazin-3-yl)-4-hydroxy-2-quinolone

Following the procedure of Example 2(a) and 1(b), except substituting 3-methyl-1-bromobutane for 2-chloromethylpyridine, the title compound was prepared as a pale yellow solid after recrystallization. MS (ES+) m/e 412 [M+H]+.

### EXAMPLE 6

1-(4-Cyanobutyl)-3-(1,1-dioxo-2H-benzo-1,2,4-thiadiazin-3-yl)-4-hydroxy-2-quinolone

Following the procedure of Example 2(a) and 1(b), except substituting 4-cyano-1-bromobutane for 2-chloromethylpyridine, the title compound was prepared as a white crystalline solid after recrystallization. MS (ES+) m/e 423 [M+H]⁺.

### EXAMPLE 7

1-(2-Butenyl)-3-(1,1-dioxo-2H-benzo-1,2,4-thiadiazin-3-yl)-4-hydroxy-2-quinolone

Following the procedure of Example 2(a) and 1(b), except substituting (E)-1-bromo-2-butene for 2-chloromethylpyridine, the title compound was prepared as a pale yellow solid after recrystallization. MS (ES+) m/e 396 [M+H]⁺.

### EXAMPLE 8

1-[(2-Methoxy)ethyl]-3-(1,1-dioxo-2H-benzo-1,2,4-thiadiazin-3-yl)-4-hydroxy-2-quinolone

Following the procedure of Example 2(a) and 1(b), except substituting 1-bromo-2-methoxy-ethane for 2-chloromethylpyridine, the title compound was prepared as a pale yellow solid after recrystallization. MS (ES+) m/e 400 [M+H]⁺.

### EXAMPLE 9

1-(n-Propyl)-3-(1,1-dioxo-2H-benzo-1,2,4-thiadiazin-3-yl)-4-hydroxy-2-quinolone

Following the procedure of Example 2(a) and 1(b), except substituting 1-bromopropane for 2-chloromethylpyridine, the title compound was prepared as a white solid after recrystallization. MS (ES+) m/e 384 [M+H]⁺.

### EXAMPLE 10

1(n-Pentyl)-3-(1,1-dioxo-2H-benzo-1,2,4-thiadiazin-3-yl)-4-hydroxy-2-quinolone

Following the procedure of Example 2(a) and 1(b), except substituting 1-bromopentane for 2-chloromethylpyridine, the title compound was prepared as a pale yellow solid after recrystallization. MS (ES+) m/e 412 [M+H]⁺.

### EXAMPLE 11

1-[(2-Methyl)propyl]-3-(1,1-dioxo-2H-benzo-1,2,4-thiadiazin-3-yl)-4-hydroxy-2-quinolone

Following the procedure of Example 2(a) and 1(b), except substituting 2-methylbromopropane for 2-chloromethylpyridine, the title compound was prepared as a solid after recrystallization. MS (ES+) m/e 398 [M+H]⁺.

### EXAMPLE 12

1-(2-Propenyl)-3-(1,1-dioxo-2H-benzo-1,2,4-thiadiazin-3-yl)-4-hydroxy-2-quinolone

Following the procedure of Example 2(a) and 1(b), except substituting allyl bromide for 2-chloromethylpyridine, the title compound was prepared as a solid after recrystallization. MS (ES+) m/e 382 [M+H]⁺.

Also included in the present invention are pharmaceutically acceptable salt complexes. Preferred are the ethylene diamine, sodium, potassium, calcium, ethanolamine, hydrochloride, hydrobromide and trifluoroacetate salts. The compounds of the present invention may contain one or more asymmetric carbon atoms and may exist in racemic and optically active forms. All of these compounds and diastereomers are contemplated to be within the scope of the present invention.

In order to use a compound of Formula (I) or a pharmaceutically acceptable salt thereof for the treatment of humans and other mammals, it is normally formulated in accordance with standard pharmaceutical practice as a pharmaceutical composition.

The present ligands can be administered by different routes including intravenous, intraperitoneal, subcutaneous, intramuscular, oral, topical, transdermal, or transmucosal administration. For systemic administration, oral administration is preferred. For oral administration, for example, the compounds can be formulated into conventional oral dosage forms such as capsules, tablets and liquid preparations such as syrups, elixirs and concentrated drops.

Alternatively, injection (parenteral administration) may be used, e.g., intramuscular, intravenous, intraperitoneal, and subcutaneous. For injection, the compounds of the invention are formulated in liquid solutions, preferably, in physiologically compatible buffers or solutions, such as saline solution, Hank's solution, or Ringer's solution. In addition, the compounds may be formulated in solid form and redissolved or suspended immediately prior to use. Lyophilized forms can also be produced.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, bile salts and fusidic acid derivatives. In addition, detergents may be used to facilitate permeation. Transmucosal administration, for example, may be through nasal sprays, rectal suppositories, or vaginal suppositories.

For topical administration, the compounds of the invention can be formulated into ointments, salves, gels, or creams, as is generally known in the art. The amounts of various compounds to be administered can be determined by standard procedures taking into account factors such as the compound (IC₅₀) potency, (EC₅₀) efficacy, and the biological half-life (of the compound), the age, size and weight of the patient, and the disease or disorder associated with the patient. The importance of these and other factors to be considered are known to those of ordinary skill in the art.

Amounts administered also depend on the routes of administration and the degree of oral bioavailability. For example, for compounds with low oral bioavailability, relatively higher doses will have to be administered. Oral administration is a preferred method of administration of the present compounds.

Preferably the composition is in unit dosage form. For oral application, for example, a tablet, or capsule may be administered, for nasal application, a metered aerosol dose may be administered, for transdermal application, a topical formulation or patch may be administered and for transmucosal delivery, a buccal patch may be administered. In each case, dosing is such that the patient may administer a single dose.

Each dosage unit for oral administration contains suitably from 0.01 to 500 mg/Kg, and preferably from 0.1 to 50 mg/Kg, of a compound of Formula (I) or a pharmaceutically acceptable salt thereof, calculated as the free base. The daily dosage for parenteral, nasal, oral inhalation, transmucosal or transdermal routes contains suitably from 0.01 mg to 100 mg/Kg, of a compound of Formula(I). A topical formulation contains suitably 0.01 to 5.0% of a compound of Formula (I). The active ingredient may be administered from 1 to 6 times per day, preferably once, sufficient to exhibit the desired activity, as is readily apparent to one skilled in the art.

As used herein, “treatment” of a disease includes, but is not limited to prevention, retardation, prophylaxis, therapy and cure of the disease. As used herein, “diseases” treatable using the present compounds include, but are not limited to keratitis, encephalitis, herpes labialis, neonatal disease, genital herpes, chicken pox, shingles, pneumonia, colitis, retinitis, cytomegalic inclusion disease, roseola, febrile seizures, bone marrow graft suppression, interstitial pneumonitis, multiple sclerosis, mononucleosis, Burkitt's lymphoma, nasopharyngeal carcinoma, Hodgkin's disease, Kaposi's sarcoma, and multiple myeloma.

Composition of Formula (I) and their pharmaceutically acceptable salts which are active when given orally can be formulated as syrups, tablets, capsules and lozenges. A syrup formulation will generally consist of a suspension or solution of the compound or salt in a liquid carrier for example, ethanol, peanut oil. olive oil, glycerine or water with a flavoring or coloring agent. Where the composition is in the form of a tablet, any pharmaceutical carrier routinely used for preparing solid formulations may be used. Examples of such carriers include magnesium stearate, terra alba, talc, gelatin, acacia, stearic acid, starch, lactose and sucrose. Where the composition is in the form of a capsule, any routine encapsulation is suitable, for example using the aforementioned carriers in a hard gelatin capsule shell. Where the composition is in the form of a soft gelatin shell capsule any pharmaceutical carrier routinely used for preparing dispersions or suspensions may be considered, for example aqueous gums, celluloses, silicates or oils, and are incorporated in a soft gelatin capsule shell.

Typical parenteral compositions consist of a solution or suspension of a compound or salt in a sterile aqueous or non-aqueous carrier optionally containing a parenterally acceptable oil, for example polyethylene glycol, polyvinylpyrrolidone, lecithin, arachis oil or sesame oil.

Typical compositions for inhalation are in the form of a solution, suspension or emulsion that may be administered as a dry powder or in the form of an aerosol using a conventional propellant such as dichlorodifluoromethane or trichlorofluoromethane.

A typical suppository formulation comprises a compound of Formula (I) or a pharmaceutically acceptable salt thereof which is active when administered in this way, with a binding and/or lubricating agent, for example polymeric glycols, gelatins, cocoa-butter or other low melting vegetable waxes or fats or their synthetic analogs.

Typical dermal and transdermal formulations comprise a conventional aqueous or non-aqueous vehicle, for example a cream, ointment, lotion or paste or are in the form of a medicated plaster, patch or membrane.

Preferably the composition is in unit dosage form, for example a tablet, capsule or metered aerosol dose, so that the patient may administer a single dose.

No unacceptable toxological effects are expected when compounds of the present invention are administered in accordance with the present invention.

The HCV NS5B inhibitory activity of the compounds of Formula (I) was determined using standard procedures well known to those skilled in the art and described in, for example Behrens et al., EMBO J. 15:12-22 (1996) and Lohmann et al., Virology 249:108-118 (1998).

All publications, including but not limited to patents and patent applications cited in this specification are herein incorporated by reference as if each individual publication were specifically and individually indicated to be incorporated by reference as though fully set forth.

What is claimed is:

1. A compound according to formula (I) hereinbelow: 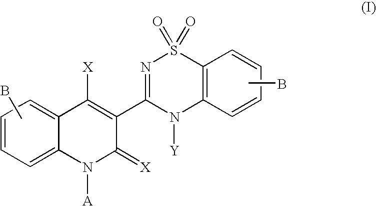
wherein:
A is selected from the group consisting of C 1-6 alkyl, C 2-6 alkenyl, alkylaryl, aryl, and heteroaryl;
B is selected from one or more of the group consisting of H, C 1-6 alkyl, C 1-6 cycloalkyl, halo, OR1, COR1, COOR1, CONR1R2, and CN wherein R1 and R2 are, independently, H, C 1-6 alkyl, aryl and heteroaryl;
X is selected from the group consisting of O, OR1, S, and SR1 wherein R1 is as defined above; and
Y is selected from the group consisting of H, C 1-6 alkyl, alkylaryl, aryl, and heteroaryl.

2. A compound according to claim 1 selected from the group consisting of:
1-(n-Propyl)-3-(1,1-dioxo-2H-benzo-1,2,4-thiadiazin-3-yl)-4-hydroxy-2-quinolone;
1-(n-Butyl)-3-(1,1-dioxo-2H-benzo-1,2,4-thiadiazin-3-yl)-4-hydroxy-2-quinolone;
1-Benzyl-3-(1,1-dioxo-2H-benzo-1,2,4-thiadiazin-3-yl)-hydroxy-2-quinolone;
1-(2-Pyridylmethyl)-3-(1,1-dioxo-2H-benzo-1,2,4-thiadiazin-3-yl)-4-hydroxy-2-quinolone;
1-[(2-Methyl)propyl)-3-(1,1-dioxo-2H-benzo-1,2,4-thiadiazin-3-yl)-4-hydroxy-2-quinolone
1-(3-Cyanopropyl)-3-(1,1-dioxo-2H-benzo-1,2,4-thiadiazin-3-yl)-4hydroxy-2-quinolone;
1-[(3-Methyl)butyl)-3-(1,1-dioxo-2H-benzo-1,2,4-thiadiazin-3-yl)-4-hydroxy-2-quinolone;
1-(4-Cyanobutyl)-3-(1,1-dioxo-2H-benzo-1,2,4-thiadiazin-3-yl)-4hydroxy-2-quinolone;
1-(n-Pentyl)-3-(1,1-dioxo-2H-benzo-1,2,4-thiadiazin-3-yl)-4-hydroxy-2-quinolone; and
1-(2-Butenyl)-3-(1,1-dioxo-2H-benzo-1,2,4-thiadiazin-3-yl)-4-hydroxy-2-quinolone.
1-(2-Propenyl)-3-(1,1-dioxo-2H-benzo-1,2,4-thiadiazin-3-yl)-4-hydroxy-2-quinolone.

3. A method of treating or preventing infection which comprises administering to a subject in need thereof, an effective amount of a compound according to claim 1.

4. A method according to claim 3 which involves inhibiting HCV.

5. A method according to claim 3 in which the compound is administered in an oral dosage form.

6. A method of preparing a compound according to claim 1 comprising the step of reacting an anhydride with an anion.
